(19) United States
(12) Patent Application Publication
(10) Pub. No.: US 2017/0259018 A1
(43) Pub. Date: Sep. 14, 2017

(54) INHALATION MASK ASSEMBLY

(71) Applicant: ACCUTRON, INC., Phoenix, AZ (US)

(72) Inventors: Richard J. Blasdell, Phoenix, AZ (US); Jonathan W. Blasdell, Phoenix, AZ (US)

(21) Appl. No.: 15/069,571

(22) Filed: Mar. 14, 2016

(51) Int. Cl. A61M 16/00; A61M 16/20; A61M 16/01; A61M 16/06

(52) U.S. Cl. CPC A61M 16/009; A61M 16/06; A61M 16/20; A61M 16/01; A61M 2230/432

(57) ABSTRACT

An inhalation mask assembly includes a cup shaped first body and a cup shaped second body. The first body is receivable against a facial area for enclosing a respiratory organ, and includes first inhalation members, and an exhalation valve assembly at a frontal midpoint of the first body between the first inhalation members. The second body includes exhalation members and second inhalation members. The first body is nested in the second body, and the first inhalation members of the first body are coupled gaseously to the respective second inhalation members. The first inhalation members are for administering respirable gas into the first body from the respective second inhalation members, the exhalation valve assembly is for exhausting exhaust gas from the first body into a scavenger chamber formed between the first body and the second body, and the exhalation members are for exhausting the exhaust gas from the scavenger chamber.

### FIELD OF THE INVENTION

The present invention relates to inhalation masks for use in the administration of medical and dental gases.

### BACKGROUND OF THE INVENTION

Inhalation or respiratory systems for administering respirable gas to an individual are well known. Of particular significance are apparatus employed in the medical and dental arts for dispensing medical and dental gases, such as anesthetic and analgesic gases and oxygen and nitrous oxide, to a patient.

Typically, inhalation systems include a source of a selected pressurized gas and a means to deliver the gas to the external respiratory organs of the patient. A breathing device, such as a mask, is fitted to the face of the patient to embrace the nose and/or the mouth. The source, which may be either portable or fixed, usually includes a flow regulator. A delivery conduit, generally in the form of a flexible hose, communicates between the pressurized gas source and the breathing device. Optionally, the system may include scavenging mechanism including a return conduit extending from the breathing device to a source of vacuum.

The prior art has devised an array of personal breathing devices in numerous structural configurations. These personal breathing devices, commonly referred to as inhalation masks or facemasks, serve a variety of functions. Known, for example, are masks that extend over the mouth and the nose of the patient while others receive only the nose. Commonly, masks include a fitting for attachment of a gas delivery conduit. In some instances, an exhalation valve is incorporated into the mask to prevent the entrance of ambient air.

In recognition of the concern over collection and disposal of expired gas, skilled artisans have developed a variety of solutions useful with inhalation masks, such as scavenger valve attachments, scavenger hoods useful with inhalation masks, and scavenger circuits. Although these prior art examples initially appeared adequate, inherent structural complexities inherent in the prior art scavenging solutions have proven less than satisfactory, thereby necessitating continued improvement in the art. Accordingly, there is a continuing need in the art for an inhalation an inhalation mask assembly that is useful for administering a respirable gas to a respiratory organ of an individual and for scavenging exhaust gas from the respiratory organ which is inexpensive, simple in structure, easy to use, and easy to assemble and disassemble without the need for specialized skill.

### SUMMARY OF THE INVENTION

According to the principle of the invention, an inhalation mask assembly for administering a respirable gas to an individual, which individual includes a facial area containing a respiratory organ for receiving respirable gas and for expelling exhaust gas, includes a cup shaped first body, and a cup shaped second body. The first body is receivable against the facial area for enclosing the respiratory organ, is integrally formed, and includes first inhalation members, and an exhalation valve assembly, the first inhalation members extend from either side of the first body, and the exhalation valve assembly is at a frontal midpoint of the first body between the first inhalation members. The second body is integrally formed and includes second inhalation members, and exhalation members, the second inhalation members and the exhalation members extend from either side of the second cup shaped body. The first body is nested in the second body, and the first inhalation members of the first body are coupled gaseously to the respective second inhalation members. The first inhalation members are for administering respirable gas into the first body from the respective second inhalation members, the exhalation valve assembly is for exhausting exhaust gas from the first body into a scavenger chamber formed between the first body and the second body, and the exhalation members are for exhausting the exhaust gas from the scavenger chamber. The inhalation mask assembly further includes a capnography monitor sample line for monitoring end-tidal CO₂ of the exhaust gas exhausted into the first body from the respiratory organ. The sample line extends through the second body, the scavenger chamber, and the first body from an outlet outside of the second body to an inlet inside the first body. The second body is transparent for enabling visualization of moisture from the exhaust gas in the scavenger chamber. The first body is nested removably in the second body.

According to the principle of the invention, an inhalation mask assembly for administering a respirable gas to an individual, which individual includes a facial area containing a respiratory organ for receiving respirable gas and for expelling exhaust gas, includes a cup shaped first body, and a cup shaped second body. The first body is receivable against the facial area for enclosing the respiratory organ, is integrally formed, and includes first inhalation members, and an exhalation valve assembly, the first inhalation members extend from either side of the first body, and the exhalation valve assembly is at a frontal midpoint of the first body between the first inhalation members. The second body is integrally formed and includes second inhalation members, and exhalation members, the second inhalation members and the exhalation members extend from either side of the second cup shaped body. The second body is carried removably by the first body, and the first inhalation members of the first body are inserted removably into the respective second inhalation members. The first inhalation members are for administering respirable gas into the first body from the respective second inhalation members, the exhalation valve assembly is for exhausting exhaust gas from the first body into a scavenger chamber formed between the first body and the second body, and the exhalation members is for exhausting the exhaust gas from the scavenger chamber. The inhalation mask assembly further includes a capnography monitor sample line for monitoring end-tidal CO₂ of the exhaust gas exhausted into the first body from the respiratory organ. The sample line extends through the second body, the scavenger chamber, and the first body from an outlet outside of the second body to an inlet inside the first body. The second body is transparent for enabling visualization of moisture from the exhaust gas in the scavenger chamber.

According to the principle of the invention, an inhalation mask assembly for administering a respirable gas to an individual, which individual includes a facial area containing a respiratory organ for receiving respirable gas and for expelling exhaust gas, includes a cup shaped first body, and a cup shaped second body. The first body is receivable against the facial area for enclosing the respiratory organ, and includes first inhalation members, and an exhalation valve assembly. The first inhalation members extend from either side of the first body, and the exhalation valve assembly is at a frontal midpoint of the first body between the first inhalation members. The second body includes second inhalation members, and exhalation members, the second inhalation members and the exhalation members extend from either side of the second cup shaped body. The first body is nested in the second body, and the first inhalation members of the first body are coupled gaseously to the respective second inhalation members. The first inhalation members are for administering respirable gas into the first body from the respective second inhalation members, the exhalation valve assembly is for exhausting exhaust gas from the first body into a scavenger chamber formed between the first body and the second body, and the exhalation members are for exhausting the exhaust gas from the scavenger chamber. The mask assembly further includes a capnography monitor sample line for monitoring end-tidal CO₂ of the exhaust gas exhausted into the first body from the respiratory organ. The sample line extends through the second body, the scavenger chamber, and the first body from an outlet outside of the second body to an inlet inside the first body. The second body is transparent for enabling visualization of moisture from the exhaust gas in the scavenger chamber. The first body is nested removably in the second body.

### BRIEF DESCRIPTION OF THE DRAWINGS

Referring to the drawings:

FIG. 1 is a front right view of an inhalation mask assembly constructed and arranged in accordance with the principle of the invention;

FIG. 2 is a front left view of the embodiment of FIG. 1;

FIGS. 3-7 are partially exploded views of the embodiment of FIG. 1;

FIGS. 8 and 9 are partially exploded fragmentary views of the embodiment of FIG. 1;

FIG. 10 is a top front view of the embodiment of FIG. 1;

FIG. 11 is a right rear view of the embodiment of FIG. 1;

FIG. 12 is a left rear view of the embodiment of FIG. 1;

FIG. 13 is a rear view of the embodiment of FIG. 1;

FIG. 14 is a top view of the embodiment of FIG. 1;

FIG. 15 is a bottom view of the embodiment of FIG. 1;

FIG. 16 is a right bottom view of the embodiment of FIG. 1;

FIG. 17 is a left bottom view of the embodiment of FIG. 1;

FIG. 18 is a left view of the embodiment of FIG. 1;

FIG. 19 is a right view of the embodiment of FIG. 1; and

FIG. 20 is a fragmentary view of the embodiment of FIG. 1 with portions thereof being broken away for illustrative purposes.

### DETAILED DESCRIPTION

The present invention provides, among other things, a new and improved inhalation mask assembly for administering or otherwise conducting a respirable gas to an individual, which individual includes a facial area having a respiratory organ for conducting respirable gas into the individual's respiratory tract and for expelling exhaust gas from the individual's respiratory tract, the exhaust gas having a moisture content or level and a carbon dioxide (CO₂) content or level when expelled from the respiratory organ in accordance with normal exhaust gas expelled from human beings.

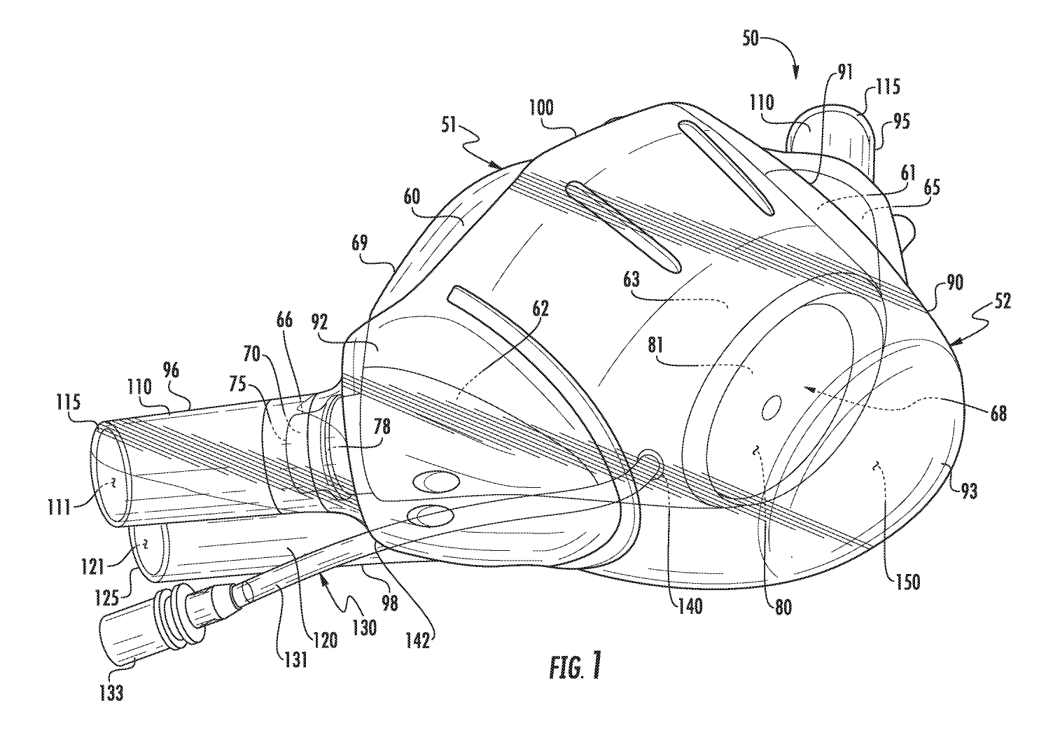

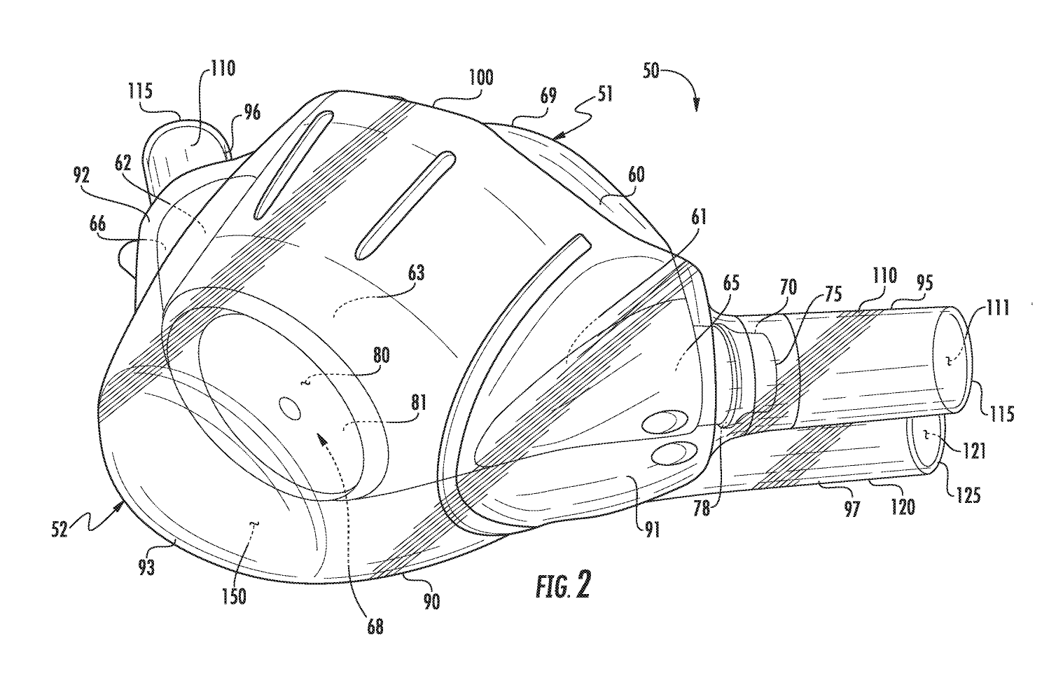

Turning now to the drawings, in which like reference characters indicate corresponding elements throughout the several views, attention is first directed to FIGS. 1 and 2 in which there is seen an inhalation mask assembly 50.

Inhalation mask assembly 50 includes a mask 51 and a scavenger 52 carried by or otherwise engaged to mask 51. In mask assembly 50, scavenger 52 not only is for receiving and conducting respirable gas to mask 52, but also is for receiving and exhausting exhaust gas from mask 52.

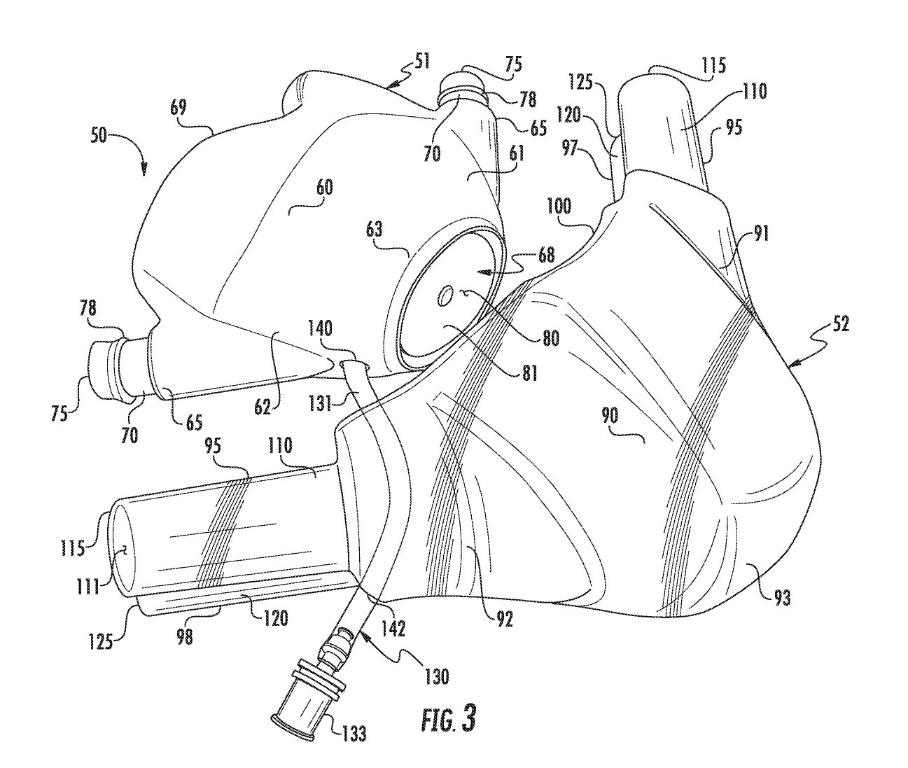

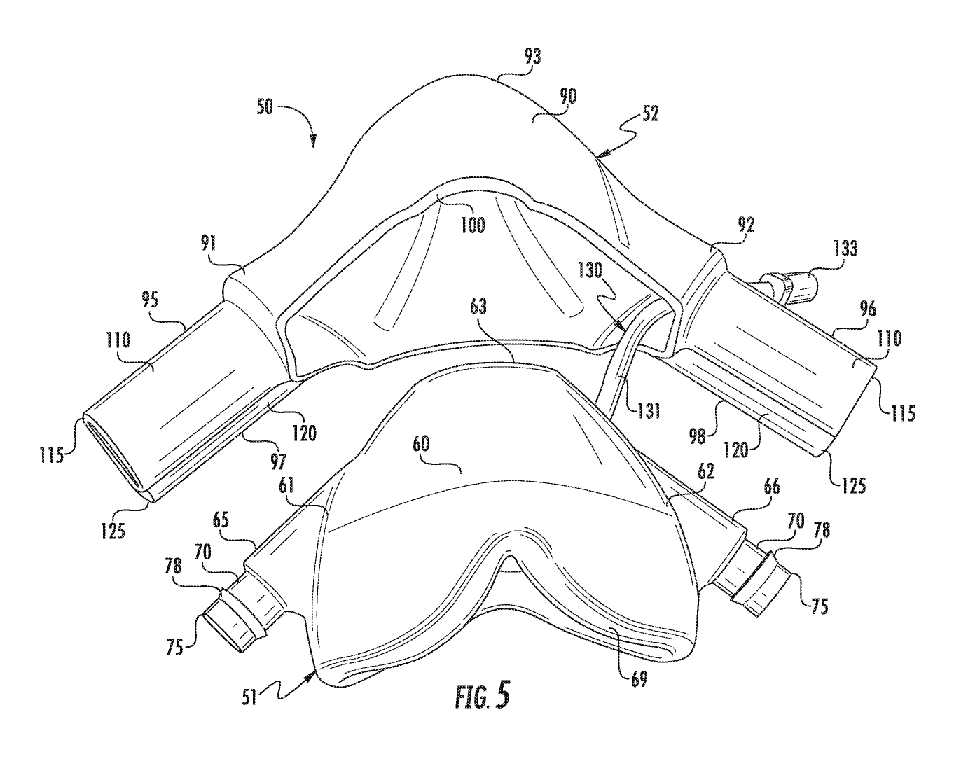

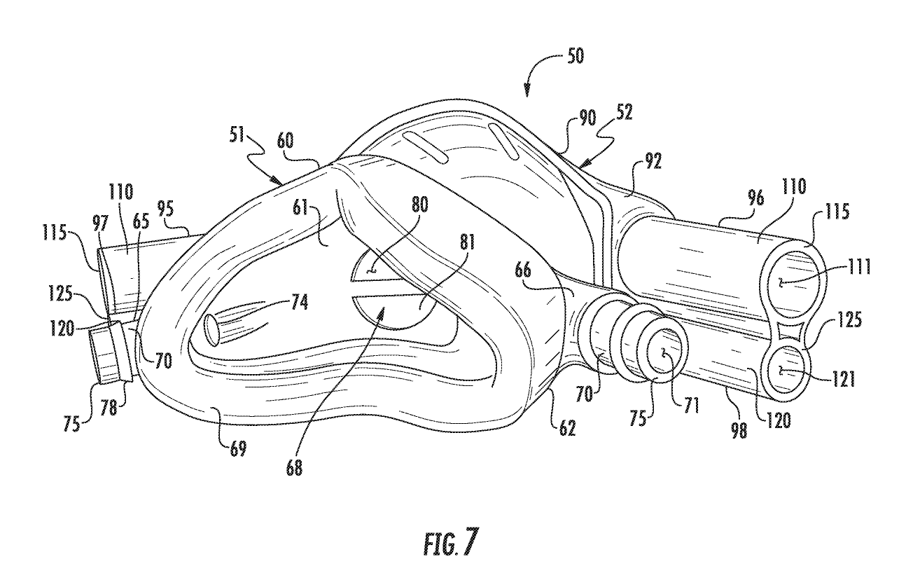

Referring to FIGS. 3-7 in relevant part, mask 51 includes a body 60. Body 60 is cup shaped, is receivable against the facial area for enclosing the respiratory organ, and is integrally formed, being a single, unitary body. Body 60 includes opposite sides 61 and 62, a frontal midpoint 63, inhalation members 65 and 66, and an exhalation valve assembly 68. Inhalation member 65 for the intake of respirable gas into mask 51 from side 61 extends from side 61 of body 60, and inhalation member 66 for the intake of respirable gas into mask 51 from side 62 extends from side 62 of body 60. Exhalation valve assembly 60 for the expiration of exhaust gas from mask 51 is at frontal midpoint 63 of body 60 between inhalation members 65 and 66. Body 60 terminates with a rearwardly directed peripheral edge 69 encircling an opening into mask 51 for receiving the respiratory organ of the individual. Edge 69 is a terminal portion of body 60 and is in-turned to form a semi-cylindrical bead. Body 60 is sized to receive the external respiratory organs of an individual, specifically the nose. Edge 69 is contoured to be received against the facial area surrounding the nose. Body 60 is molded of a flexible elastic material such as the thermoplastic elastomer found under the trademark KRATON®. Edge 69 is readily deformable, which enables it to be pliantly received against the facial area in sealing engagement therewith.

Each one of inhalation members 65 and 66 is a cylindrical sidewall 70 having a bore 71 extending therethrough defined by cylindrical sidewall 70 from outlet 74 to inlet 75. Inhalation members 65 and 66 each project angularly rearward from either side of body 60 from outlet 74 to inlet 75. Each cylindrical sidewall 70 has an annular gasket 78 between outlet 74 and inlet 75. Each gasket 78 is continuous, circumscribes the corresponding cylindrical sidewall 70, and projects radially outwardly from the corresponding continuous sidewall 70. Respirable gas is applied through bore 71 of each one of inhalation members 65 and 66 from inlet 75 to outlet 74.

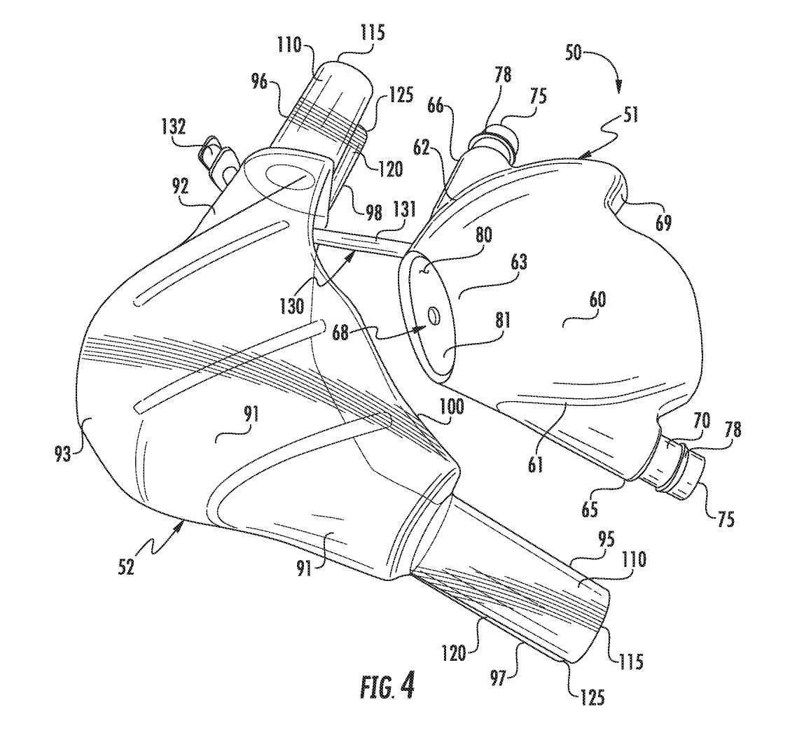

In FIGS. 3 and 4, exhalation valve assembly 68 is a conventional and well-known flapper valve assembly, consisting of outlet port 80 formed with a valve disc 81, a relatively thin resiliently deformable member that is also be fabricated of a thermoplastic material. Valve disc 81 is drawn into sealing engagement with a valve seat of outlet port 80 disabling gas from passing into mask 51 through outlet port 80, when the individual inhales in mask 51. Valve disc 81 deflects away from the valve seat of outlet port 80 enabling exhaust gas to evacuate mask 51 through outlet port 80, when the individual exhales in mask 51. Valve disc 81 and outlet port 80 function as an outlet check valve for unidirectional flow of expired gas from mask 51. Again, exhalation valve assembly 68 is known and well-known, further details of will readily occur to the skilled artisan and are not discussed in further detail.

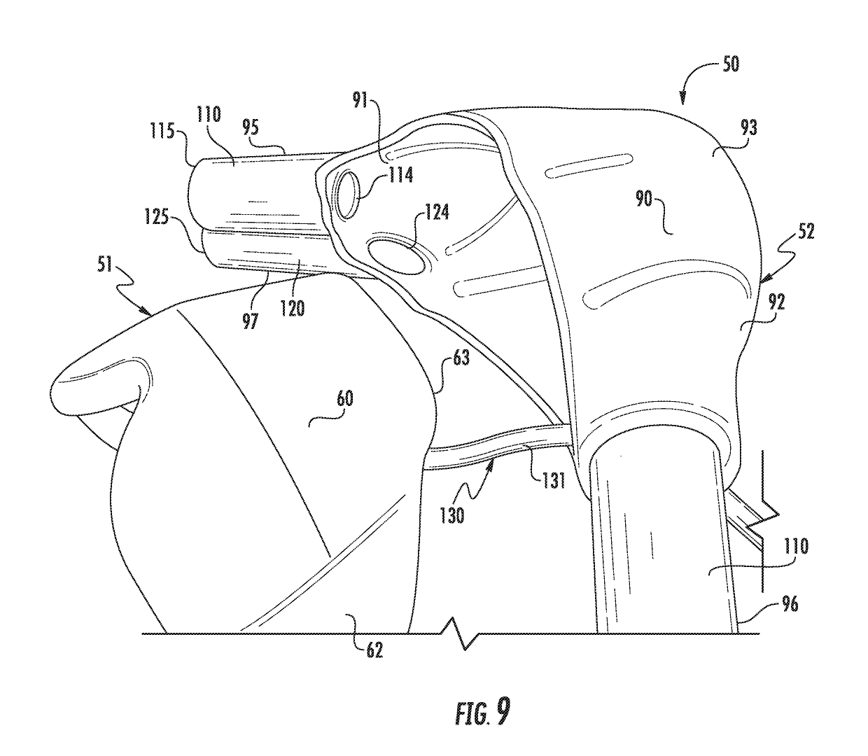

Referring in relevant part to FIGS. 3-9, scavenger 52 includes a body 60. Body 90 is cup shaped, and is integrally formed, being a single, unitary body. Body 90 includes opposite sides 91 and 92, a frontal midpoint 93, inhalation members 95 and 96, and exhalation members 97 and 98. Inhalation member 95 for the intake or respirable gas from side 91 extends from side 91 of body 90, inhalation member 96 for the intake of respirable gas from side 92 extends from side 92 of body 90, exhalation member 97 for the outtake of exhaust gas from side 91 extends from side 91 of body 90, and exhalation member 98 for the outtake of exhaust gas from side 92 extends from side 92 of body 90. Body 69 terminates with a rearwardly directed peripheral edge 100 encircling an opening into scavenger 52 for receiving mask 51 front midpoint 63 first. Edge 100 is a terminal portion of body 90. Body 90 is sized to receive mask 51. Body 90 is molded of a flexible elastic material, which is transparent, such as the thermoplastic elastomer found under the trademark KRATON®.

Each one of inhalation members 95 and 96 is a cylindrical sidewall 110 having a bore 111 extending therethrough defined by cylindrical sidewall 110 from outlet 114 to inlet 115. Respirable gas is applied through bore 111 of each one of inhalation members 95 and 96 in a direction from inlet 115 to outlet 114. Inhalation members 95 and 96 each project angularly rearward from body 60 from either side of body 90 from outlet 114 to inlet 115. Each one of exhalation members 97 and 98 is a cylindrical sidewall 120 having a bore 121 extending therethrough defined by cylindrical sidewall 120 from inlet 124 to outlet 125. Exhalation members 97 and 98 each project angularly rearward from body 60 from either side of body 90 from inlet 124 to outlet 125. Exhaust gas is applied through bore 121 of each one of exhalation members 97 and 98 from inlet 124 to outlet 125.

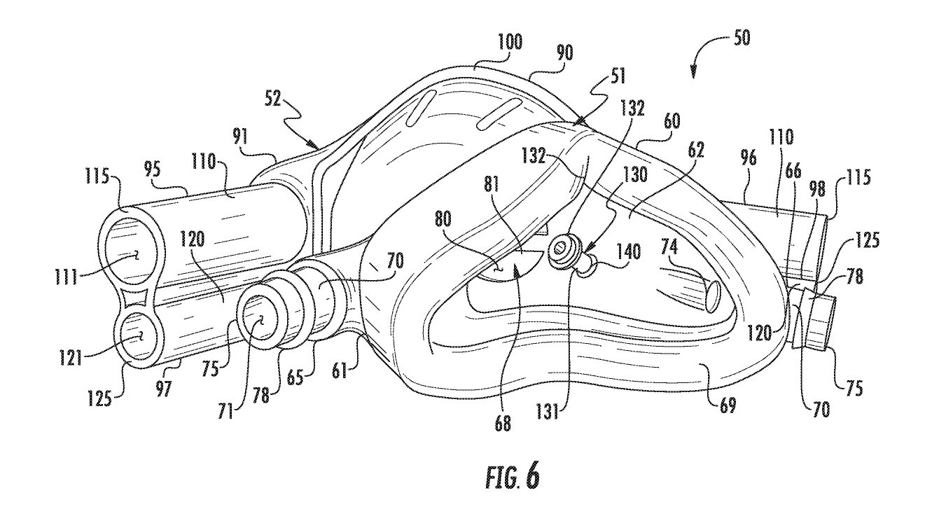

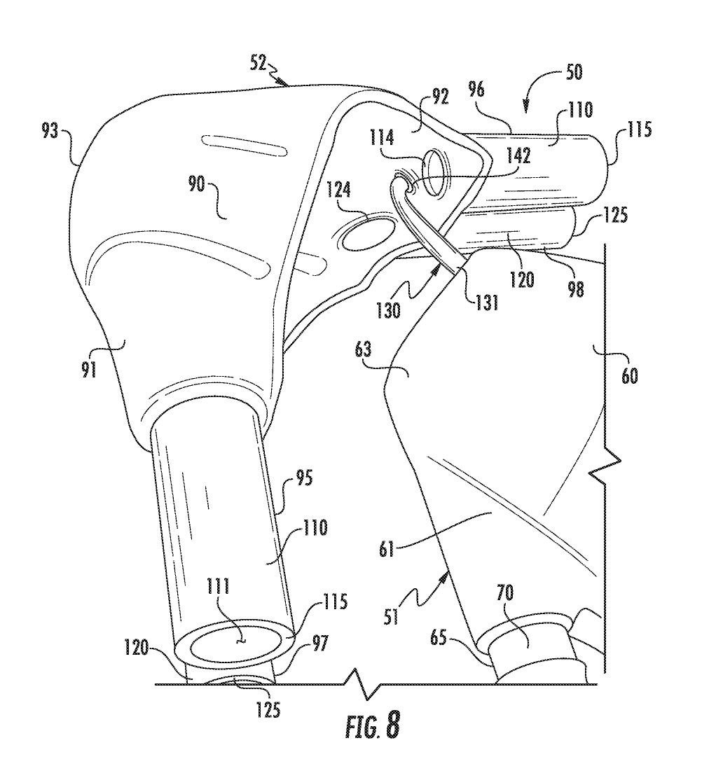

In FIGS. 3-6, mask assembly 50 further includes a capnography monitor sample line 130 useful for monitoring end-tidal CO₂ of the exhaust gas exhausted into mask 51 from the respiratory organ. Sample line 130, a flexible conduit 131, includes an inlet 132 and an outlet 133. In FIGS. 3 and 6, an opening 140 extends through body 60 of mask 51 at side 62 of body 60 between exhalation valve assembly 68 and outlet 74 of inhalation member 66. In FIGS. 3 and 8, an opening 142 extends through body 90 of mask scavenger 52 at side 92 of body 90 between outlet 114 of inhalation member 96 and inlet 124 of exhalation member 98. Conduit 131 extends from outlet 133 outside of scavenger 52 and into body 90 of scavenger 52 through opening 142, and from body 90 of scavenger 52 through opening 140 to inlet 132 in body 60 of mask 51. Inlet 132 is enlarged, which disables it form being withdrawn outwardly from opening 140.

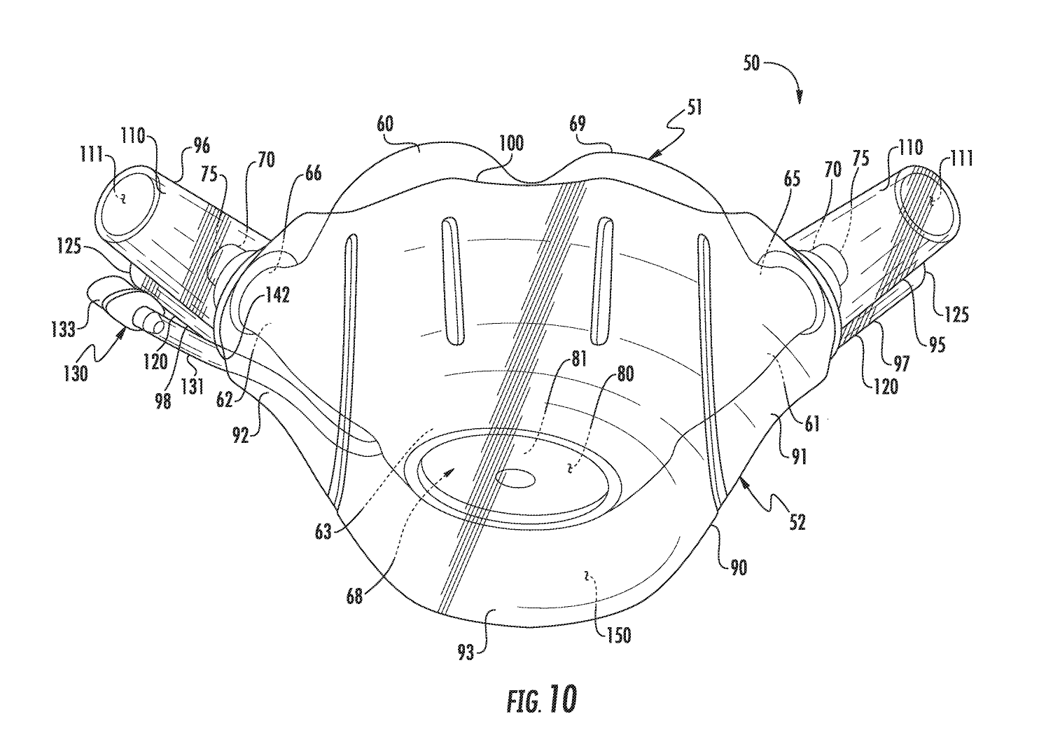

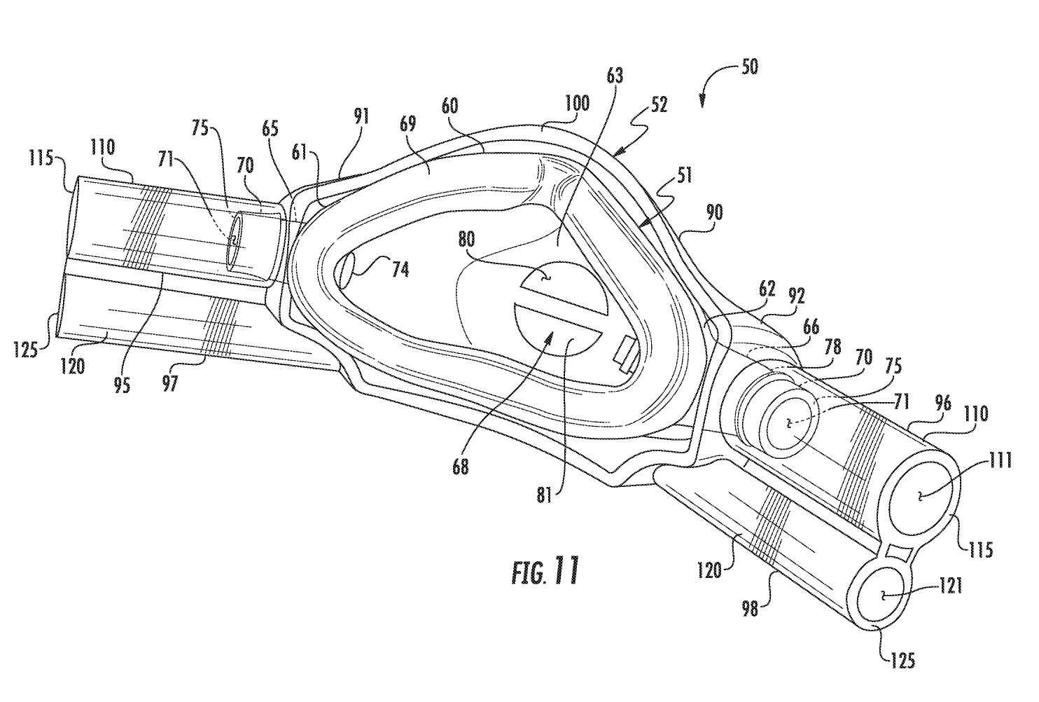

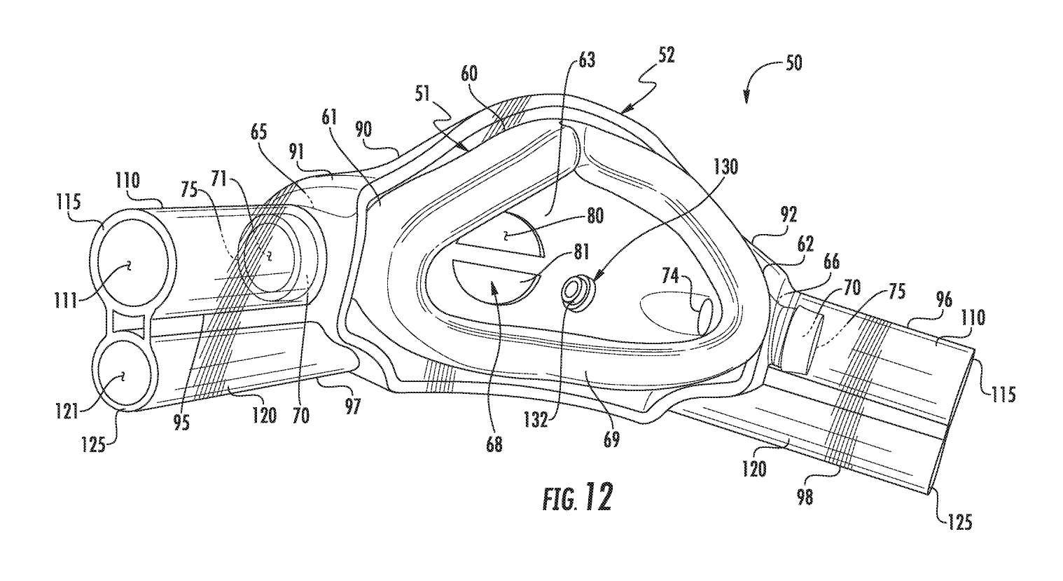

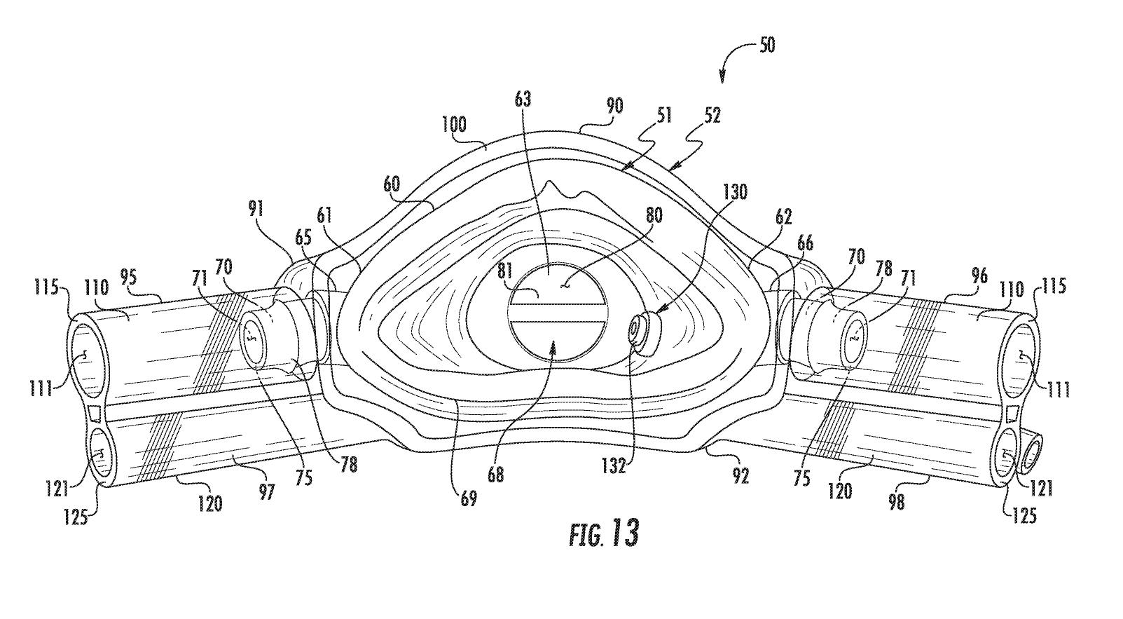

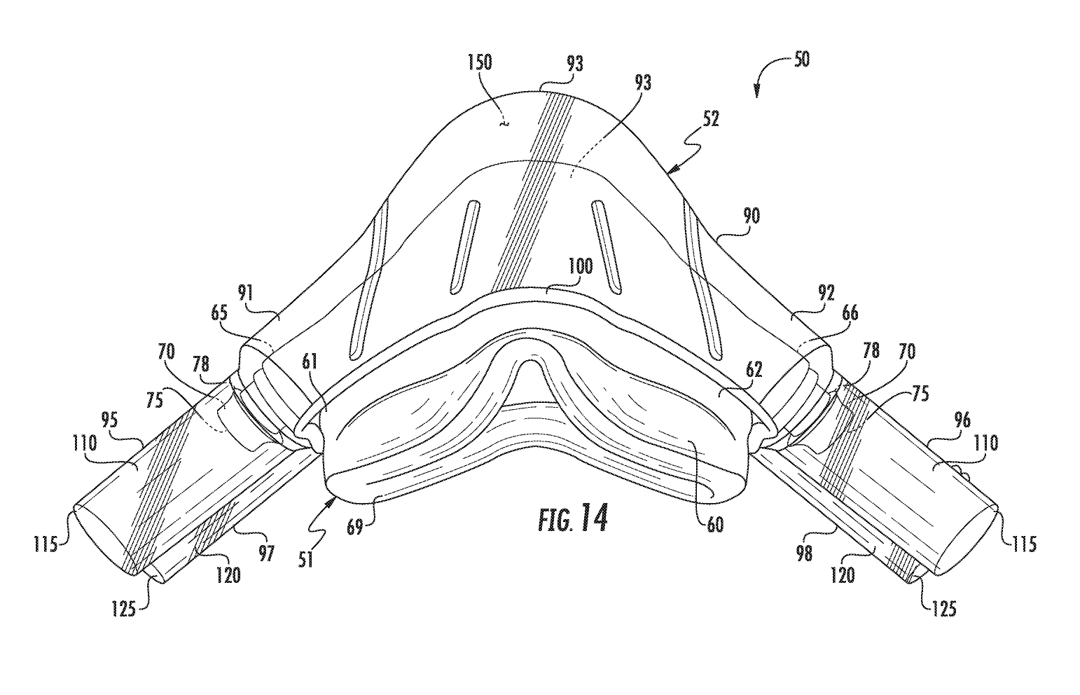

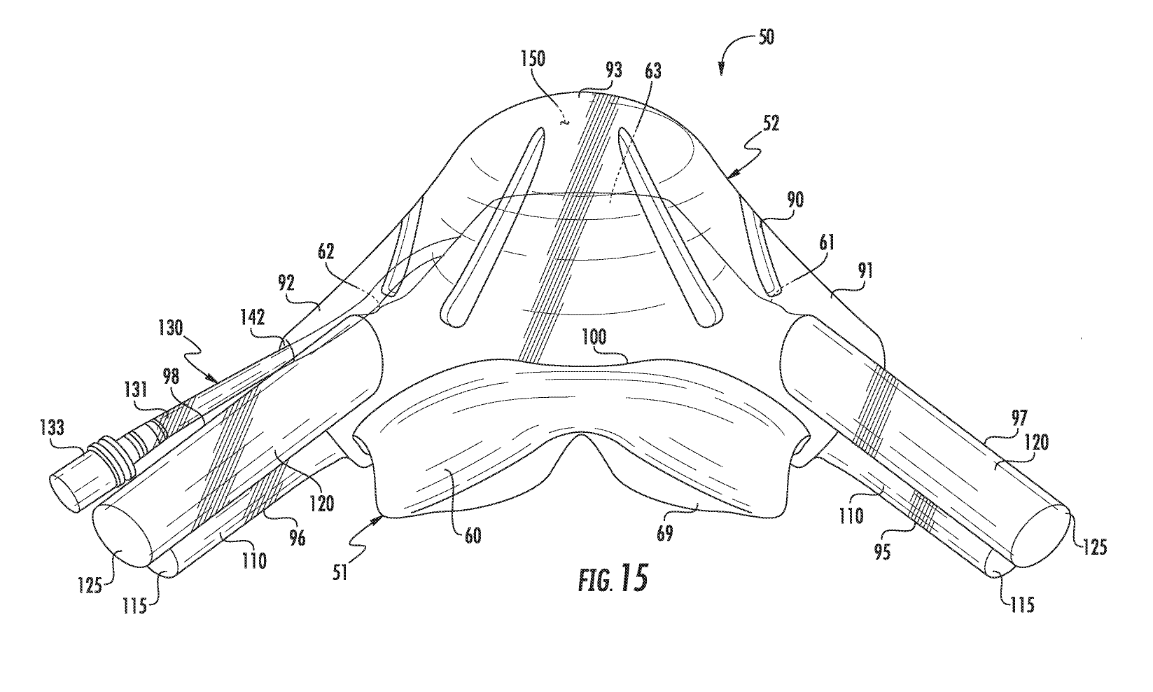

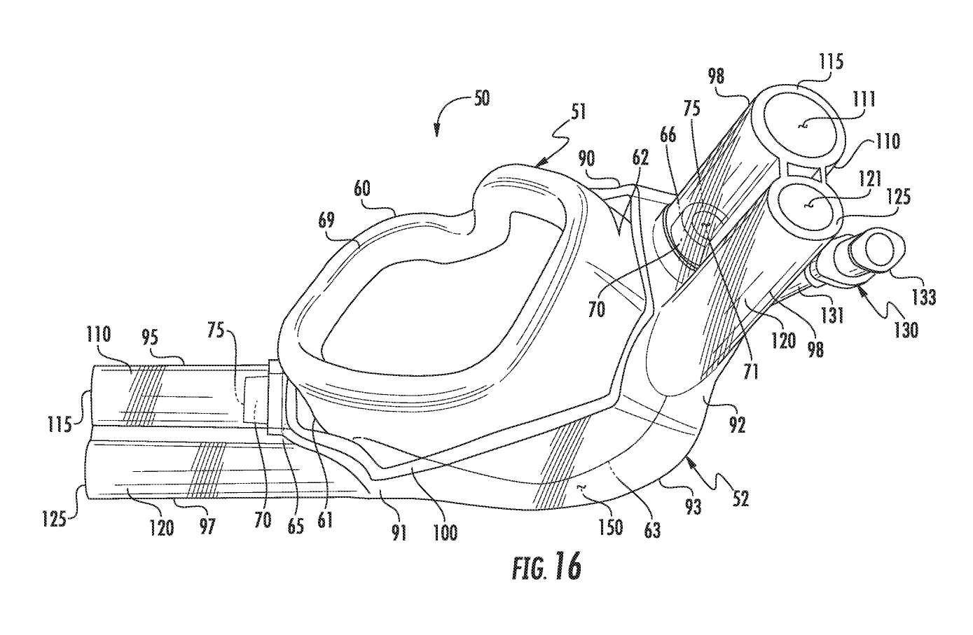

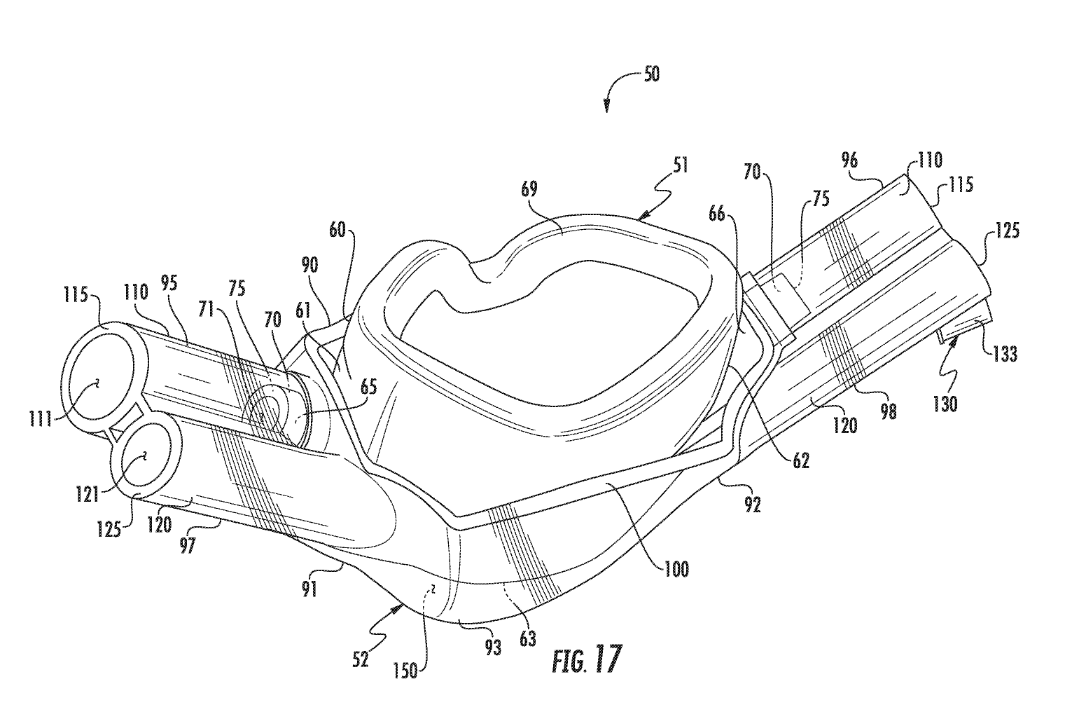

Scavenger 52 is carried by mask 51 in FIGS. 1, 2, and 10-19. In FIGS. 1, 2, and 10-19, the lead lines of some reference numerals incorporate dashed lines, which indicates that the lead line is pointing through transparent body 90 to a structural feature.

Referring to FIGS. 1, 2, and 10-19 in relevant part, body 60 of mask 51 is coupled releasably to body 90 of scavenger 52. Body 60 of mask 51 is nested removably in body 90 of scavenger 52, and inhalation members of body 60 are coupled gaseously to the respective inhalation members 95 and 96 of body 90 of scavenger 52. Inhalation members 65 and 66 are for administering a respirable gas into body 60 of mask 51 from the respective inhalation members 95 and 96 of body 90 of scavenger 52, exhalation valve assembly 68 is for exhausting exhaust gas from body 60 of mask 51 into a scavenger chamber 150 formed between body 60 of mask 51 and body 90 of scavenger 52, and exhalation members 97 and 98 of body 90 of scavenger 52 are for exhausting the exhaust gas from scavenger chamber 150. Sample line 130 extends through body 90 of scavenger 52, scavenger chamber 150, and body 60 of mask 51 from outlet 133 outside of body 90 of scavenger 52 to inlet 132 inside body 60 of mask 51.

Exhaust valve assembly 68 of body 60 of mask 51 at frontal midpoint 63 confronts frontal midpoint 93 of body 90 of scavenger 52 to define a space, namely, scavenger chamber 150. Inlets 75 of inhalation members 65 and 66 extend into the respective bores 71 through the respective outlets 114 of the respective inhalation members 95 and 96 of body 90 of scavenger 52.

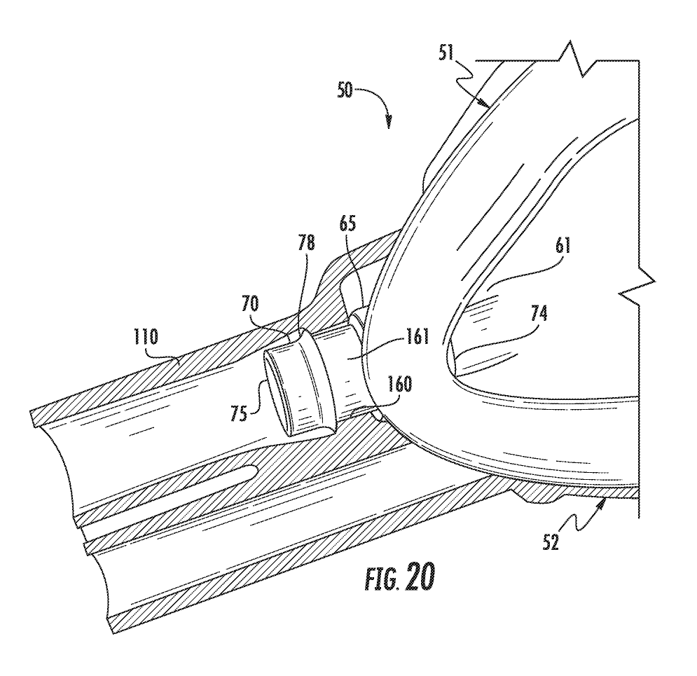

Looking briefly to FIG. 20, in the installation of scavenger 52 to mask 51, inlet 75 of inhalation member 65 is inserted removably into bore 71 through outlet 114 of inhalation member 95. An annular rib 160 extends inwardly from the inner diameter of cylindrical sidewall 110 into an annular seat 161 of the outer diameter of cylindrical sidewall 70 between gasket 78 and the outer side of body 60 of mask 51. This arrangement secures inhalation member 65 of mask 51 releasably to inhalation member 95 of scavenger 52, and gasket 78 applied between bore 71 and cylindrical sidewall provides a seal therebetween. Inlet 75 of inhalation member 66 is identically installed into bore 71 through outlet 114 of inhalation member 96. To separate scavenger 52 from mask 51, the two parts need only be pulled apart, such as by hand, to withdraw inhalation members 65 and 66 from the respective inhalation members 95 and 96 and to withdrawn separate scavenger 52 from mask 51.

In mask assembly 50, respirable gas is introduced into bores 111 of inhalation members 95 and 96 of body 90 of scavenger 52 through inlets 115. The respirable gas, in turn, passes into bores 71 of inhalation members 65 and 66 of mask 51 through the respective inlets 75 from inhalation members 95 and 96 of scavenger 52 and into body 60 from either side thereof through outlets 74. As the individual breaths through his respiratory organ, his nose, the respirable gas is conducted into the individual's respiratory tract via his nose, and exhaust gas is expelled into body 60 of mask 51 from the individual's respiratory tract via his nose. Of course, the exhaust gas inherently has a moisture content or level and a carbon dioxide (CO₂) content or level when expelled from the respiratory organ in accordance with normal exhaust gas expelled from human beings. Exhalation valve assembly 68 exhausts the exhaust gas from body 60 into scavenger chamber 150 formed between body 60 of mask 51 and body 90 of scavenger 52. The exhaust gas exhausted into scavenger chamber 150 from body 60 of mask 51 is, in turn, exhausted from scavenger chamber 150 through bores 121 of exhalation members 97 and 98 from inlets 124 and outwardly through outlets 125.

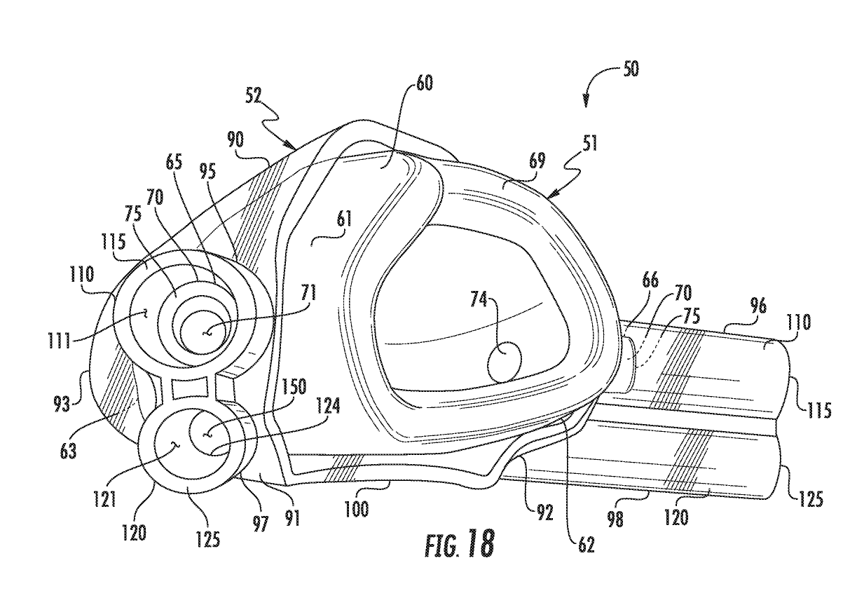

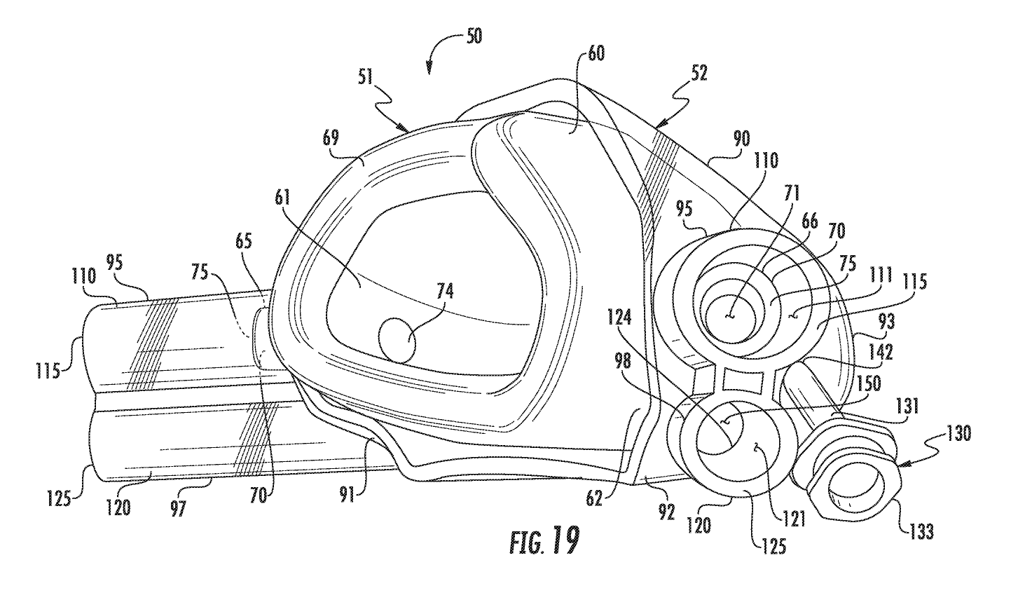

Inlets 115 of inhalation members 95 and 96 of body 90 of scavenger 52 can be coupled to a respirable gas source for applying the respirable gas to body 60 of mask 51. Outlets 125 of exhalation members 97 and 98 of body 90 of scavenger 52 can, in turn, be coupled to vacuum scavenger apparatus for withdrawing exhaust gas from scavenger chamber 150 through exhalation members 97 and 98. The assembly of inhalation members 65 and 66 to the respective inhalation members 95 and 96 conduct the respirable gas into mask 51 for inhalation by the individual. Upon exhalation by the individual, exhalation valve assembly 68 will conduct exhaled gas from 51 and into scavenger chamber 150. Once in scavenger chamber 150, the vacuum scavenger source will pull the exhaled gas outwardly from scavenger chamber 150 through exhalation members 97 and 98. In FIG. 18, scavenger chamber 150 is seen through bore 121 of exhalation member 97. In FIG. 19, scavenger chamber 150 is seen through bore 121 of exhalation member 98.

Capnography is the monitoring of the concentration or partial pressure of CO₂ in exhaust gas. Its main development has been as a monitoring tool for use during anesthesia and intensive care. It is usually presented as a graph of expiratory CO₂ (measured in millimeters of mercury, “mmHg”) plotted against time, or, less commonly, but more usefully, expired volume. Side tube with Lure Lock port allows connection to a capnography monitor's sample line for monitoring patient end-tidal CO₂. In mask assembly 50, outlet 133 located on the outside of body 90 of scavenger 52 can be coupled to a capnography monitor for drawing end-tidal CO₂ of exhaust gas exhausted into body 60 of mask 51 from the respiratory organ of the individual through sampling line from inlet 132 in body 60 of mask 51 to outlet 133 and from outlet 133 to the capnography monitor for monitoring the individual's end-tidal CO₂.

As explained above, body 90 of scavenger 52 is transparent, which enables an observer to look into scavenger chamber 150. During use of mask assembly 50, the moisture content contained in the exhaust gas exhaled into scavenger chamber 150 by the user will condense upon the inner surface of body 90 of scavenger 52, which enables a doctor, dentist, or observer to look through body 90 of scavenger 52 into scavenger chamber 150 and observe whether the user of mask assembly 50 is breathing properly by virtue of noticing whether condensation is forming on inner surface of body 90 of scavenger 52.

As described above, mask 51 carries scavenger 52, and scavenger 52 is coupled removably to mask 51. Mask 51 can be disposable, so as to be designed to be discarded after a single use. Scavenger 52 can also be disposable, so as to be designed to be discarded after a single use. If desired, scavenger 52 can be reused after suitable cleaning between uses such as by autoclaving.

The invention has been described above with reference to illustrative embodiments. However, those skilled in the art will recognize that changes and modifications may be made to the embodiments without departing from the nature and scope of the invention. Various changes and modifications to the embodiments herein chosen for purposes of illustration will readily occur to those skilled in the art. To the extent that such modifications and variations do not depart from the spirit of the invention, they are intended to be included within the scope thereof.

Having fully described the invention in such clear and concise terms as to enable those skilled in the art to understand and practice the same, the invention claimed is:

1. An inhalation mask assembly for administering a respirable gas to an individual, which individual includes a facial area containing a respiratory organ for receiving respirable gas and for expelling exhaust gas, the inhalation mask assembly comprising:
a cup shaped first body;
a cup shaped second body;
the first body is receivable against the facial area for enclosing the respiratory organ, is integrally formed, and includes first inhalation members, and an exhalation valve assembly, the first inhalation members extend from either side of the first body, and the exhalation valve assembly is at a frontal midpoint of the first body between the first inhalation members;
the second body is integrally formed and includes second inhalation members, and exhalation members, the second inhalation members and the exhalation members extend from either side of the second cup shaped body;
the first body is nested in the second body, and the first inhalation members of the first body are coupled gaseously to the respective second inhalation members; and
the first inhalation members are for administering respirable gas into the first body from the respective second inhalation members, the exhalation valve assembly is for exhausting exhaust gas from the first body into a scavenger chamber formed between the first body and the second body, and the exhalation members are for exhausting the exhaust gas from the scavenger chamber.

2. The inhalation mask assembly according to claim 1, further comprising a capnography monitor sample line for monitoring end-tidal CO₂ of the exhaust gas exhausted into the first body from the respiratory organ, the sample line extends through the second body, the scavenger chamber, and the first body from an outlet outside of the second body to an inlet inside the first body.

3. The inhalation mask assembly according to claim 1, wherein the second body is transparent for enabling visualization of moisture from the exhaust gas in the scavenger chamber.

4. The inhalation mask assembly according to claim 1, wherein the first body is nested removably in the second body.

5. An inhalation mask assembly for administering a respirable gas to an individual, which individual includes a facial area containing a respiratory organ for receiving respirable gas and for expelling exhaust gas, the inhalation mask assembly comprising:
a cup shaped first body;
a cup shaped second body;
the first body is receivable against the facial area for enclosing the respiratory organ, is integrally formed, and includes first inhalation members, and an exhalation valve assembly, the first inhalation members extend from either side of the first body, and the exhalation valve assembly is at a frontal midpoint of the first body between the first inhalation members;
the second body is integrally formed and includes second inhalation members, and exhalation members, the second inhalation members and the exhalation members extend from either side of the second cup shaped body;
the second body is carried removably by the first body, and the first inhalation members of the first body are inserted removably into the respective second inhalation members; and
the first inhalation members are for administering respirable gas into the first body from the respective second inhalation members, the exhalation valve assembly is for exhausting exhaust gas from the first body into a scavenger chamber formed between the first body and the second body, and the exhalation members is for exhausting the exhaust gas from the scavenger chamber.

6. The inhalation mask assembly according to claim 5, further comprising a capnography monitor sample line for monitoring end-tidal CO₂ of the exhaust gas exhausted into the first body from the respiratory organ, the sample line extends through the second body, the scavenger chamber, and the first body from an outlet outside of the second body to an inlet inside the first body.

7. The inhalation mask assembly according to claim 5, wherein the second body is transparent for enabling visualization of moisture from the exhaust gas in the scavenger chamber.

8. An inhalation mask assembly for administering a respirable gas to an individual, which individual includes a facial area containing a respiratory organ for receiving respirable gas and for expelling exhaust gas, the inhalation mask assembly comprising:
a cup shaped first body;
a cup shaped second body;
the first body is receivable against the facial area for enclosing the respiratory organ, and includes first inhalation members, and an exhalation valve assembly, the first inhalation members extend from either side of the first body, and the exhalation valve assembly is at a frontal midpoint of the first body between the first inhalation members;
the second body includes second inhalation members, and exhalation members, the second inhalation members and the exhalation members extend from either side of the second cup shaped body;
the first body is nested in the second body, and the first inhalation members of the first body are coupled gaseously to the respective second inhalation members;
the first inhalation members are for administering respirable gas into the first body from the respective second inhalation members, the exhalation valve assembly is for exhausting exhaust gas from the first body into a scavenger chamber formed between the first body and the second body, and the exhalation members are for exhausting the exhaust gas from the scavenger chamber; and
a capnography monitor sample line for monitoring end-tidal CO₂ of the exhaust gas exhausted into the first body from the respiratory organ, the sample line extends through the second body, the scavenger chamber, and the first body from an outlet outside of the second body to an inlet inside the first body.

9. The inhalation mask assembly according to claim 8, wherein the second body is transparent for enabling visualization of moisture from the exhaust gas in the scavenger chamber.

10. The inhalation mask assembly according to claim 8, wherein the first body is nested removably in the second body.
